(19) United States
(12) Patent Application Publication
(10) Pub. No.: US 2009/0232834 A1
(43) Pub. Date: Sep. 17, 2009

(54) Methods and Agents to Treat Autoimmune Diseases

(76) Inventors: Saleh A. Al-Harbi, Al-Salem (KW); David D. Haines, Bloomfield, CT (US)

Correspondence Address: Pearl Cohen Zedek Latzer, LLP, 1500 Broadway, 12th Floor, New York, NY, 10036, US

(21) Appl. No.: 11/989,011

(22) PCT Filed: Jul. 18, 2006
(86) PCT No.: PCT/US06/27903

§ 371 Date: Mar. 24, 2009

Related U.S. Application Data
(60) Provisional application No. 60/667,030, filed on Jul. 18, 2005.

(51) Int. Cl. A61K 39/00; A61K 35/12; C12N 5/06; A61P 37/02

(52) U.S. Cl. 424/184.1; 424/93.7; 435/325

(57) ABSTRACT

A therapeutic method for preventing, suppressing, or treating an autoimmune disease is described. This method involves administering to a patient suffering from an autoimmune disease an effective amount of a composition containing an allogeneic or autologous leucocyte cell population derived from a healthy donor. The composition is administered by subcutaneous injection and induces an immunological response in recipient patients sufficient to reduce incidence, prevalence, frequency, or severity of the autoimmune disease.

### FIELD OF THE INVENTION

The present invention relates to embodiments of methods of treating an autoimmune disease in a subject. Specifically, provided herein is a method of alloimmunization or autoimmunization of subjects afflicted by an autoimmune disease with compositions containing allogeneic or autologous leukocytes and secretions thereof, derived from healthy individuals, or cells derived from cell lines, and treated ex-vivo to enhance therapeutic benefit.

### BACKGROUND OF THE INVENTION

A variety of T cells are involved in the cell-mediated response. Some induce particular B cell clones to proliferate and produce antibodies specific for the antigen. Others recognize and destroy cells presenting foreign antigens on their surfaces. Certain T cells regulate the response by either stimulating or suppressing other cells.

While the normal immune system is closely regulated, aberrations in immune response are not uncommon. In some instances, the immune system functions inappropriately and reacts to a component of the host as if it were, in fact, foreign. Such a response results in an autoimmune disease, in which the hosts immune system attacks the host's own tissue. T cells, as the primary regulators of the immune system, directly or indirectly affect such autoimmune pathologies.

Numerous diseases are believed to result from autoimmune mechanisms. Prominent among these are rheumatoid arthritis, systemic lupus erythematosus, multiple sclerosis, Type I diabetes, myasthenia gravis and pemphigus vulgaris. Autoimmune diseases affect millions of individuals world-wide and the cost of these diseases, in terms of actual treatment expenditures and lost productivity, is measured in billions of dollars annually. At present, there are no known effective treatments for autoimmune pathologies. Usually, only the symptoms can be treated, while the disease continues to progress, often resulting in severe debilitation or death.

Immunotherapies suggested by others in the field have partially or completely failed to treat several diseases which are known to be caused by autoimmune disorders. These therapies included the use of antibodies, chimeric proteins or DNA vaccination, drugs based on combination of interferon beta and glatiramer acetate, approaches based on induction of non-responsiveness in allergen-specific T-helper 2 cells by allergen peptides or redirection of allergen-specific T-helper 2 responses by T-helper 1-inducing cytokines or altered peptide ligands, approaches involving induction of peripheral tolerance or immunotoxin targeting of activated T cells and cytokine manipulations and the use of peptide-based tolerance induction.

Thus, a long-felt need exists for an effective means of curing or ameliorating autoimmune mediated pathologies. Such a treatment should ideally control the inappropriate T cell and B cell responses, rather than merely reduce the symptoms.

It is towards improving the effectiveness of mononuclear cell alloimmunization and autoimmunization for the treatment of autoimmune diseases that the present invention is directed.

### SUMMARY OF THE INVENTION

In one embodiment, provided herein is a method for treating an autoimmune disease in a subject comprising the step of administering to said subject a composition comprising stressed mononuclear cells, wherein said stressed mononuclear cells induce tolerance to self-antigens, thereby treating the autoimmune disease.

In another embodiment, provided herein is a method for treating diabetes in a subject, comprising administering to said subject a composition comprising thermally stressed mononuclear cells, wherein said thermally stressed mononuclear cells induce tolerance to self antigens.

In one embodiment, provided herein is a method for treating rheumatoid arthritis (RA), psoriasis or their combination in a subject, comprising administering to said subject a composition comprising thermally stressed mononuclear cells, wherein said thermally stressed mononuclear cells induce tolerance to self antigens.

In another embodiment, provided herein is a method for treating uveitis in a subject, comprising administering to said subject a composition comprising thermally stressed mononuclear cells, wherein said thermally stressed mononuclear cells induce tolerance to self antigens.

In one embodiment, provided herein is a vaccine for the treatment of an autoimmune disease comprising thermally stressed mononuclear cells, wherein said thermally stressed mononuclear cells induce tolerance to self-antigens.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 compares the change in post-prandial blood glucose levels in type I diabetic patients that underwent treatment in accordance with the invention, to the change in patients not treated, two months following treatment with injection vehicle-treated patients. Data set A shows the effect using live, stressed allogeneic cells; data set B, heat-killed, stressed allogeneic cells; data set C, live, stressed autologous cells; and data set D, placebo; and

FIGS. 2A-2B shows the effect of treatment in accordance with the invention on major indicators of disease severity in rheumatoid arthritis patients, as compared to untreated patients Indicators included erythrocyte sedimentation rate (ESR; FIG. 2A) and serum rheumatoid factor (RF; FIG. 2B).

### DETAILED DESCRIPTION OF THE PRESENT INVENTION

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

Higher organisms are characterized by an immune system that affords protection against invasion by potentially deleterious substances or microorganisms. When a substance, termed an antigen, enters the body and is recognized as foreign, the immune system mounts both an antibody-mediated response and a cell-mediated response. Cells of the immune system termed B lymphocytes, or B cells, produce antibodies that specifically recognize and bind to the foreign substance. Other lymphocytes termed T lymphocytes, or T cells, both effect and regulate the cell-mediated response resulting eventually in the elimination of the antigen. Under certain circumstances, the immune system functions inappropriately and reacts to a component of the host as if it were, in fact, foreign. Such a response results in an auto immune disease, in which the host's immune system attacks the host's own tissue. T cells, as the primary regulators of the immune system, directly or indirectly affect such autoimmune pathologies.

In one embodiment, provided herein is a therapeutic method for preventing, suppressing, or treating an autoimmune response which involves administering to patients suffering from an autoimmune disease an effective amount of a composition including one or more allogeneic leukocyte cell populations, and their secretions, obtained from one or more healthy donors. Leukocyte cell populations may also be obtained from the patient himself (autologous) or from cell lines. The leukocyte (monocyte) cell populations are subjected to particular stress conditions ex vivo (or in vitro in the case of cell lines) preceding or following a period of culture in certain embodiments. Following stress and culturing in one embodiment, the cells are typically killed, for example by freeze-thaw, prior to administration. The compositions of this invention induce an immunological response in recipient patients sufficient to reduce incidence, prevalence, frequency or severity of at least one immune mediated disorder.

Autologous mononuclear cells are taken in one embodiment from a subject suffering from an autoimmune disease as described herein, exposed to stress conditions as described herein and are administered to the subject according to the methods described herein, using in one embodiment, the vaccines described herein.

Therefore, in one embodiment, provided herein is a method for treating an autoimmune disease in a subject comprising the step of administering to said subject a composition comprising stressed mononuclear cells, wherein said stressed mononuclear cells induce tolerance to self-antigens, thereby treating an autoimmune disease.

The allogeneic leukocytes are cultured in one embodiment ex vivo prior to administration and are enriched in mononuclear cells or lymphocytes in another embodiment, including secretions thereof. The administration of allogeneic leukocytes, which may be killed in one embodiment, are treated in accordance with the embodiments described herein is done in one embodiment by subcutaneous injection once monthly for a period of about four months.

Autoimmune diseases result from conditions in which one's own tissues are subject to deleterious effects of the immune system, wherein a specific humoral or cell-mediated immune response is launched in the body against its own tissues. This invention describes a novel immunotherapeutic approach to treatment of this disorder. While Applicants have no duty to disclose the theory by which the invention operates, and the invention is not bound to any such disclosure, the immunotherapeutic treatment, as disclosed herein, includes induction of tolerance to self-antigens through the administration of stressed allogeneic or autologous leukocytes, which may show beneficial effects in both humans and animal models.

In one embodiment, provided herein is a method of alloimmunization or autoimmunization in another embodiment, so as to prevent or control specific T cell or B cell mediated pathologies, including autoimmune diseases and the unregulated replication of T cells. Alloimmunization is achieved by administration of a composition comprising allogeneic leukocytes, or, in another embodiment lymphocytes, or mononuclear cells in other embodiments. Allogeneic leukocytes are cultured prior to simultaneously with or after stress induction in one embodiment, and may be killed and administered in other embodiments.

The composition of allogeneic or autologous leukocytes comprises natural secretions of a variety of leukocytes. These secretions may comprise, for example, cytokines, hormones, interleukins, or a combination thereof. The composition is administered to a patient in a manner that induces an immune response directed against the leukocyte mediated pathology. The induced immune response, for example, down regulates or deletes the pathogenic T cells or B cells, thus ablating disease pathogenesis.

As used herein, the term “cytokine” refers to any one of the numerous factors that exert a variety of effects on cells, for example, inducing growth or proliferation. Nonlimiting examples of cytokines which may be used alone or in combination in the practice of the present invention include, interleukin-2 (IL-2), stem cell factor (SCF), interleukin 3 (IL-3), interleukin 6 (IL-6), interleukin 12 (IL-12), G-CSF, granulocyte macrophage-colony stimulating factor (GM-CSF), interleukin-1 alpha (IL-1-alpha), interleukin 11 (IL-11), MIP-1-alpha, leukemia inhibitory factor (LIF), c-kit ligand, thrombopoietin (TPO) and flt3 ligand.

In another embodiment, there is provided a method of treating autoimmune disorders through alloimmunization with mononuclear cells. Using the protocol developed by the inventors herein and in one embodiment, subjects suffering from a variety of autoimmune disorders are treated successfully through administration of allogeneic leukocytes.

Methods, uses, and compositions described herein, may be used for preventing and/or inhibiting chronic immune mediated disorders including hyperactive immune responses such as asthma/allergies and autoimmune diseases. Such autoimmune diseases may include conventional organ specific autoimmunity, neurological disease, rheumatic diseases/connective tissue disease, autoimmune cytopenias, and related autoimmune diseases. Such conventional organ specific autoimmunity may include thyroiditis, gastritis, adrenalitis (Addison's disease), ovaritis, primary biliary cirrhosis, myasthenia gravis, gonadal failure, hypoparathyroidism, alopecia, psoriasis, malabsorption syndrome, pernicious anemia, hepatitis, anti-receptor antibody diseases, hypopituitarism, diabetes insipidus, sicca syndrome and multiple sclerosis.

Rheumatic diseases or connective tissue diseases that may be treated by the method of this invention include rheumatoid arthritis, systemic lupus erythematosus (SLE) or Lupus, scieroderma, polymyositis, inflammatory bowel disease, dermatomyositis, ulcerative colitis, Crohn's disease, vasculitis, psoriatic arthritis, Reiter's syndrome, exfoliative psoriatic dermatitis, pemphigus vulgaris, Sjorgren's syndrome. Other autoimmune related diseases that may be treated as described herein include autoimmune uveitis, glomerulonephritis, post myocardial infarction cardiotomy syndrome, pulmonary hemosiderosis, amyloidosis, sarcoidosis, aphthous stomatitis, and other immune related diseases, as presented herein and known in the related art.

There are multiple sequelae to chronic immune mediated disorders. As non-limiting examples, autoimmunity can result in end organ failure or cancer. Chronic inflammation, as occurs in chronic immune mediated disorders, can cause the release of molecules like serum amyloid which can cause pathology. Serum amyloid is associated with amyloidosis peripherally and senile dementia in the central nervous system. In one embodiment, provided herein is a low or non toxic composition to prevent disease in asymptomatic subjects without the need to screen them for risk of developing chronic immune mediated disorders. In another embodiment, the compositions described herein, made according to the methods described herein may also be used in certain subpopulations at higher risk for developing the disorders than others.

According to a typical embodiment of this invention, as disclosed herein, there is provided a method of treating patients suffering from insulin-dependent diabetes mellitus (IDDM). IDDM is a disease resulting from autoimmune ablation of the beta islet cells of the pancreas, which normally are the primary source of insulin, in a process mediated primarily by T lymphocyte subsets with receptor specificity for antigens expressed on islet tissue. Novel immunotherapeutic approaches to treatment of this disorder include induction of tolerance to these antigens and vaccination with allogeneic or in another embodiment, autologous leukocytes treated in accordance to the methods described herein, both of which are shown herein to have certain beneficial effects in both humans and animal models.

As will be seen in the example below, the results from a double blind clinical trial of forty-three IDDM patients given subcutaneous injections of peripheral blood mononuclear cells isolated from close relatives and stressed using the embodiments of the stressing regimens described herein, demonstrated a significant recovery of these patients as compared to control patients. The control group consisted of forty-two age and sex-matched IDDM patients treated with placebo. Changes in body weight, serum c-peptide and blood sugar level were measured at the time of initial vaccination and again at four weeks thereafter. Significant changes in each of the variables measured were noted in patients receiving allogeneic leukocytes but not in the placebo-treated group. Increased serum c-peptide (p=0.001), increased body weight (p=0.001) and lowered postprandial blood sugar were observed in the test group. Results of this study indicate that leukocyte vaccination in accordance with the methods described herein provides an effective general approach to autoimmune disorders.

“Treating” embraces in another embodiment, the amelioration of an existing condition. The skilled artisan would understand that treatment does not necessarily result in the complete absence or removal of symptoms. Treatment also embraces palliative effects: that is, those that reduce the likelihood of a subsequent medical condition. The alleviation of a condition that results in a more serious condition is encompassed by this term. Therefore, in one embodiment, provided herein is a method of treating or ameliorating complications of autoimmune disorder involving the skin. According to the invention, as described herein, patients afflicted with psoriasis vulgaris were treated with allogeneic lymphocytes treated ex vivo in accordance with the methods described herein. The treatment encompassed four injections of treated allogeneic lymphocytes, in a monthly interval (one injection per month during four months of treatment). An exceptional clearing of the lesions, after the first injection, and a complete recovery after four months were evident in all patients undergoing this treatment.

In one embodiment, the term “treatment” refers to any manner in which one or more of the symptoms of a disease or disorder are ameliorated or otherwise beneficially altered. Treatment also encompasses any pharmaceutical use of the compositions and methods described herein, such as use for treating an autoimmune-mediated diseases or disorders, or diseases or disorders in which sensitivity to self antigens are related.

Immunotherapy, according to the methods of the invention disclosed herein, also has been shown to be effective in patients suffering from alopecia areata. Patients suffering from this disease were alloimmunized with lymphocytes isolated from a healthy individual and then subjected to stress conditions ex vivo. After from about 1 to about four months of therapy, and about one to two injections per month, patients' hair started to grow and the baldness was completely cured. In a more typical embodiment of this invention, the healthy individuals are close relatives of the patient.

According to another embodiment of the invention, women suffering from recurrent spontaneous abortion, and or female autoimmune infertility, are alloimmunized with stressed allogeneic or autologous lymphocytes obtained from a healthy individual donor or pool of healthy donors. The therapy is shown to treat patients completely and enabled them to carry babies to maturity. In a more typical embodiment of this invention, healthy individual donors are the husbands or the male companion of these patients. According to another embodiment, women seeking to conceive through in vitro fertilization are alloimmunized with stressed mononuclear cells obtained from a healthy individual donor or pool of donors. In one embodiment, the therapy allows retention of the conceptus by patients and normal pregnancy. In an embodiment of this invention, healthy individual donors of mononuclear cells are the husbands.

According to another embodiment of this invention, treatment is provided to patients suffering from Hashimoto's autoimmune thyroiditis, Ig nephropathy, pemphigus vulgaris and systemic lupus erythematosus. In all these cases, the disease was either substantially ameliorated or completely cured upon completion of the treatment according to this invention.

According to another embodiment of the invention, the claimed method may be administered to a large number of mammals with different risks for developing a given chronic immune disorder and only certain subpopulations may be shown to benefit statistically from the administration. The subpopulations may include mammals at higher risk than the general population. Non-limiting examples of the subpopulations include those with family history of at least one chronic immune mediator disorder, those who are deemed at high risk because of genetic or biochemical screening of themselves or of their biological relatives, those at risk because of an abnormal birth as in prematurity or small size, and those at risk because of an environmental impact or disaster such as a country's civilian population after war. For a review on an autoimmune disease-associated study see, for example, Alharbi et al., European J. of Immunogenetics: 21, 295-300 (1994), content of which is incorporated herein by reference in its entirety.

For the purpose of the present invention, the allogeneic leukocyte cell population, either as isolated or enriched for mononuclear cells or lymphocytes, is provided in an amount effective to achieve its intended purpose. In addition to the leukocytes, the composition may contain suitable pharmaceutically acceptable carriers, such as excipients, carriers and/or auxiliaries which facilitate processing of cells into preparations which can be used pharmaceutically. Such carriers are preferably adjuvants that release allogeneic leukocytes in vivo over a prolonged period. Non-limiting examples of such adjuvants are known vaccine adjuvants or depot adjuvants. Preferably the depot adjuvant comprises aluminum, calcium or salts thereof, such as aluminum sulfate (alum), aluminum phosphate, calcium phosphate or aluminum hydroxide. See, e.g., Gregoriades, G. et al., Immunological Adjuvants and Vaccines, Plenum Press, New York, 1989; Michalek S. M. et al., Cur. Sop. Microbiol. Immunol 146:5-1-58 (1989), all incorporated herein by reference. Another non-limiting example of a typical carrier is one that target macrophages and/or activates them, such as a liposome. Adjuvants that activate macrophages may be added to carriers to increase their ability to activate macrophages.

Leukocyte compositions according to the present invention may also include suitable solutions for administration intramuscularly, intravenously, subcutaneously, intradermally, orally, mucosally, or rectally or by any other type of administration. Compositions which can be administered rectally include suppositories or gels, for example. Compositions for parenteral administration include, for example, sterile aqueous or non-aqueous solutions, suspensions, and emulsions, which may contain auxiliary agents or excipients, such as suitable adjuvants, which are known in the art.

Typical modes of administration of the composition of this invention are by subcutaneous, intradermal and intravenous application. Most typical is subcutaneous administration.

It is understood that the concentration and dosage of the allogeneic leukocyte composition of the present invention administered in vivo or in vitro will be dependent upon the age, sex, health, and weight of the recipient, kind of concurrent treatment, if any, frequency of treatment, and the nature of the effect desired. The ranges of effective doses provided below are not intended to limit the invention, but represent typical dose ranges. However, the most typical dosage will be tailored to the individual subject, as is understood and determinable by one of skill in the art without undue experimentation.

In the context of the present invention “one dose” may include concurrent or separate administration of more than one allogeneic or in certain embodiments, autologous leukocyte cell population in the composition of this invention. The total dose, as in a pharmaceutically acceptable dose, required for each treatment may be administered by multiple doses or in a single dose. An allogeneic or autologous leukocyte composition may be administered alone or in conjunction with other therapeutics directed to immunologic disorders, such as allergies, immune mediated cancers and autoimmune pathologies, as known in the art.

Effective amount of pharmaceutically acceptable dosages of at least one leukocyte cell population or composition of the present invention, is from about twenty million to about six hundred million leukocytes per each administration. More preferably, the composition of the invention comprises from about thirty million to about four hundred million leukocytes per each administration. Most preferably, the composition of the invention comprises from about forty million to about two hundred million leukocytes per each administration.

In the practice of the present invention, mononuclear cells are isolated from a whole blood sample obtained from a donor, placed under particular conditions ex vivo, optionally killed, then introduced into the subject or patient suffering from an autoimmune disorder. Details of the separate procedures are described below.

1. Diagnosis: Following initial diagnosis of an individual with an autoimmune disorder, the patient is offered therapy in accordance with the present invention. The patient may present with any one of the autoimmune disorders described herein.

2. Donor Selection. If allogeneic mononuclear cells are used for treatment, patients are asked to designate a donor from whom peripheral blood leukocytes will be taken, cultured in certain embodiments, and subjected to stress conditions in accordance with the disclosure herein. In one embodiment this is the patient's spouse, but any individual may donate. Cells from cell lines may also be used. Where possible, the donor is screened for presence of bloodborne pathogens. If autologous mononuclear cells are used in one embodiment, the donor is the subject themselves.

3. Phlebotomy and Preparation of Cells for Optional Culture. Depending on the disorder, the donor is asked to undergo phlebotomy 1, 2, or 3 days prior to treating of the patient. Forty, 100 or 250 ml of whole peripheral venous blood is then sterilely collected from the donor with anticoagulant. Typically the blood is layered onto Ficoll/Hypaque separation medium and centrifuged to isolated the mononuclear cell layer, which is washed in Ringers Lactate IV and resuspended in cell culture medium (both Ham's F10 and RPMI 1640 have been used). Other procedures for isolating mononuclear cells may be employed. All procedures are conducted under sterile conditions.

Typically, blood is layered onto sterile Ficoll-Hypaque centrifugation gradient medium in 50 ml conical tubes, is centrifuged at 2,500 rpm for 45 minutes at room temperature (RCF=1500), then the entire interface is collected and wash 3× in RPMI 1640 cell culture medium. The cell density is adjusted to ˜1.5×10⁶ cells/ml with RPMI 1640, supplemented with 10% fetal calf serum (FCS).

4. Optional cultivation. The cell suspension prepared as above may optionally be placed in sterile, 500 ml culture flasks and incubated at 5% CO₂, 95% humidity at one of the following three (3) alternate conditions: about 37° C. 24 hours; 37° C. 48 hours; 37° C. 72 hours. Cultivation may precede, coincide or follow application of stress conditions. It would be readily recognized by the skilled artisan, that the conditions described hereinabove are not exact and may be altered without deviating from the scope of the invention, so long as they do not result in stressing the cells.

5. Stress conditions. In certain embodiments, one of five alternate applications of stress to which the cells are subjected are listed below. As noted above, cells optionally are cultured prior to stress treatment, or can be cultured after stress treatment, or both.

a. Flasks containing cells are not cultured at 37° C., 5% CO₂. Instead, immediately following purification they are resuspended in injection vehicle (Ringers Lactate IV) or cell culture medium (such as Hams F10 or RPMI 1640) and immersed in a hot water bath for 1 hour. Temperatures used for clinical preparation range from about 40-45° C., and timeframes from 30 minutes to 1 hour. Optimal effects are seen when the temperature is 42° C. and the time of exposure is 40 minutes.

b. Flasks containing cells are cultured at 37° C., 5% CO₂ for 47 hours. During the final hour of culture they are immersed in a hot water bath. Temperatures used for clinical preparation range from about 40-45° C., and timeframes from 30 minutes to 1 hour. Optimal effects are seen when the temperature is 40° C. and the time of culture is 30 minutes.

c. Flasks containing cells are cultured at 37° C., 5% CO₂ for 48 hours. During the culture they are agitated constantly on a rocker table, rocking, by way of non-limiting example, at 15 oscillations per minute.

d. Flasks containing cells are cultured at 37° C., 5% CO₂ for 47 hours. Cultures are supplemented with 0.05% absolute ethanol.

e. Flasks containing cells are cultured at 37° C., 5% CO₂ for 24 hours. Temperature is then reduced to 4° C. and maintained for an additional 24 hours.

f. Flasks containing cells are cultured at 20-25° C., 5% CO₂ for about 24-72 hours. In one embodiment, in a temperature of 22° C.

Each of the above embodiments of culture and stress conditions, when used in conjunction with other portions of the protocol described herein, has resulted in measurable clinical effects in treatment of autoimmune disease.

6. Cell harvest, killing and vaccine preparation. After application of stress, both the nonadherent (lymphocyte in one embodiment) and adherent (macrophage/monocyte) fractions of the cultures are harvested and washed and resuspended in Ringers Lactate IV. All procedures are conducted under sterile conditions.

In one embodiment, after culture under one of the conditions described herein, monocyte/macrophage are scraped from the bottom of flask with sterile cell scraper. Next, the entire contents are transferred in another embodiment to 50 ml conical tubes along with an equal volume of Ringers Lactate IV, then centrifuge to pellet at 2000 rpm for 10 minutes. In one embodiment, the cells are killed by freeze-thaw. The cell pellet is resuspended in sterile Ringer's lactate IV, then prepared for one of three alternative injection protocols:

a. The cell pellet is resuspended in four ml of Ringer's Lactate IV. This is administered subcutaneously in eight injections of 500 microliters per injection. Four such injections are administered to the patient in each forearm.

b. The cell pellet is resuspended in one ml of Ringer's Lactate IV. This is administered subcutaneously in four injections of 250 microliters per injection. Four such injections are administered to the patient in only one forearm; or two in each forearm.

c. Injection protocol three (used in one embodiment for treatment of recurrent spontaneous abortion). The cell pellet is resuspended in one ml of Ringer's Lactate IV. The entire four ml are divided into two aliquots: (1) one ml; and (2) three ml. The one ml aliquot is administered subcutaneously in each forearm, in eight injections of 125 microliters per injection. The injections are spaced approximately one inch apart. Next, the three ml aliquot is administered intravenously.

7. Baseline patient evaluation. On the day of the first treatment, the patient is given a baseline clinical evaluation for comparison with disease status on subsequent days.

8. Vaccination. Cell preparations are vaccinated into patient subcutaneously. Typically, the entire cell preparation harvested and suspended in Ringers Lactate IV) is divided into 8 equal aliquots, four of each are injected subcutaneously into each of both forearms. All procedures are conducted under sterile conditions.

9. Subsequent Vaccinations and Clinical Monitoring. At intervals of 4-8 weeks, the donor and patient return for booster treatments (the optimal time between treatments has not been determined, but is probably about 6 weeks). Each booster treatment involves repetition of steps 4-8 above. Selected disease parameters are recorded at the time of each treatment.

10. Outcome. Typically, most patients receiving alloimmunization in accordance with the teachings herein show substantial improvement of prognosis within a month following the vaccination. Boosters are provided for up to 8 months, however if a patient is refractory to the therapy after 3 treatments, he/she is advised to discontinue.

In one embodiment, if the mononuclear cells are placed in culture prior to subjecting the cells to stress conditions, various culture conditions can be used, such as but not limited to: 37 C for 24 hours; 37 C for 48 hours; 37 C for 72 hours. However, placing the mononuclear cells in culture is optional and is an embodiment of the present invention.

Cultivation optionally can be carried out in a CO₂ incubator, exposing the cells to about 5% CO2. In another embodiment, various stress conditions can be employed in the practice of the invention. In one embodiment, stress conditions includes exposing cells to a temperature of about 40 to about 45 C for about 30 minutes to about 1 hour. In a typical embodiment, cells are exposed to a temperature of about 42 C for about 40 minutes.

In another embodiment, stress conditions include exposing cells to a temperature of about 22 C for about 24 hours, or 22 C for 48 hours; or 22 C for 72 hours in other embodiments.

In another embodiment, the mononuclear cells are not cultured but exposed to a temperature of about 40 to about 45 C for about 30 minutes to about 1 hour. In a typical embodiment, the cells are exposed to a temperature of about 42 C for about 40 minutes. In yet another embodiment, the mononuclear cells are cultured at a temperature of about 37 C for about 47 hours, then stressed at a temperature of about 40 to about 45 C for about 30 minutes to about 1 hour. In a typical embodiment, the mononuclear cells are stressed at a temperature of about 40 C. for about 30 minutes.

In one embodiment of the invention, the mononuclear cells are cultured at a temperature of about 37 C for about 24 hours, during which time they are continuously agitated on a rocker table at about 15 oscillations per minute. Alternately, the mononuclear cells are cultured at a temperature of about 37 C in the presence of about 0.05% absolute ethanol. In another embodiment, the mononuclear cells are cultured at a temperature of about 37 C for about 24 hours, then stressed at a temperature of about 4 C for about 24 hours.

If the mononuclear cells are killed before administering to the subject, the mononuclear cells can be killed by freezing. Alternate means for killing the cells are also embraced herein, such as in one embodiment, physical means such as pressure, sonication, jet impingement, chemical means and the like in other embodiments.

In one embodiment, mononuclear cells are treated ex vivo in accordance to the embodiments of methods for stressing or treating the cells described above, or treated in vitro if the mononuclear cells are obtained from a cell line, then killed in another embodiment, prior to administration (vaccination) to the subject. In one embodiment, the mononuclear cells are administered to the subject by the subcutaneous, intradermal, or intraperitoneal routes, but it is not so limiting as to route. In one embodiment, administration is subcutaneous. In another embodiment of treatment regimen, mononuclear cells treated as described herein; are administered to the subject at four to eight weekly intervals. Typically a subject will receive about 3-4 administrations.

In another embodiment, the invention is directed to the use of optionally killed, ex-vivo-stressed allogeneic or autologous mononuclear cells or cell lines for the preparation of a medicament for administration to a subject to treat an autoimmune disease or disorder. The mononuclear cells used for the practice of the invention can be obtained from the subject, obtained from a donor different from the subject, or obtained from a cell line. In an alternate embodiment, a combination of any two or all of the foregoing sources of mononuclear cells may be employed.

In one embodiment, the term “autologous” refers to cells that are derived from the same individual or subject or cell line. In another embodiment, the term “autolgous” refers to embodiments involving one subject as both donor and recipient. The term “allogeneic” refers in one embodiment to a cell population that are genetically different although belonging to or obtained from the same species or cell line. In another embodiment, the term “allogeneic” refers to the difference in genetic loci between strains or species.

In another embodiment, a process for preparing mononuclear cells for administration to a subject to treat an autoimmune disease is provided, the process comprising the steps of (a) obtaining a population of mononuclear cells; (b) optionally maintaining the mononuclear cells in culture; (c) subjecting the mononuclear cells to stress conditions; and (d) administering the mononuclear cells to the subject in need thereof. In one embodiment, the mononuclear cells are killed prior to step (d). The mononuclear cells used for the practice of the invention can be obtained from the subject, obtained from a donor different from the subject, or obtained from a cell line. In an alternate embodiment, a combination of any two, or all of the foregoing sources of mononuclear cells, may be employed.

In one embodiment, provided herein is a method for treating diabetes in a subject, comprising administering to said subject a composition comprising thermally stressed mononuclear cells, wherein said thermally stressed mononuclear cells induce tolerance to self antigens. In another embodiment, the thermal stress to which the mononuclear cells are exposed to, comprises exposing the mononuclear cells to temperature of between about 40 to about 45° C., for a period of about 30 minutes.

In another embodiment, provided herein is a method for treating rheumatoid arthritis (RA), psoriasis or their combination in a subject, comprising administering to said subject a composition comprising thermally stressed mononuclear cells, wherein said thermally stressed mononuclear cells induce tolerance to self antigens. In another embodiment, the thermal stress to which the mononuclear cells are exposed to, comprises exposing the mononuclear cells to temperature of between about 20 to about 25° C., for a period of about 72 hours.

In one embodiment, provided herein is a method for treating uveitis in a subject, comprising administering to said subject a composition comprising thermally stressed mononuclear cells, wherein said thermally stressed mononuclear cells induce tolerance to self antigens. In another embodiment, the thermal stress to which the mononuclear cells are exposed to, comprises exposing the mononuclear cells to temperature of between about 40 to about 45° C., for a period of about 40 minutes, followed by 48 hours at 37° C.

It would be readily understood by the skilled artisan, that the thermal stressing regimen to which the mononuclear cells are exposed to, may be changed to optimize the efficacy of the treatment methods described herein, for the autoimmune diseases described herein without exceeding the scope of the invention. Thermal stress of the mononuclear cells described herein, include in another embodiment the exposure of the cells to cold temperatures, referring in one embodiment to any temperature below the body temperature of the recipient through administration. The exposure may also be to freezing temperatures, which will not kill the cells in one embodiment. These thermal stress conditions may be obtained through careful ramping down of temperature which will ensure that ice formed inside the cell will not grow to the point where it perforates cell membranes.

In one embodiment, thermal stress may be combined with other stress methods, such as pH, ionic strength, mechanical stress and the like and their combination. In one embodiment, the profile of heat-shock proteins created as the result of the stress conditions imposed on the cells induces tolerance to self antigens, or in another embodiment, the concentration of a single HSP induces the tolerance to self antigens and is used for the treatment of autoimmune diseases as described herein.

The term “heat shock protein” as used herein refers to any protein which exhibits increased expression in a cell when the cell is subjected to a stress. The heat shock protein may be a modified heat shock protein, wherein the heat shock protein has been modified to provide it with advantageous characteristics such as increased resistance to degradation or to reduce the size of a heat shock protein while still maintaining its ability to enhance the production of one or more chemokines in the cells of a recipient. Heat shock proteins (hsps) are highly conserved proteins that play an important role in various cellular processes. Hsps are stress proteins that are typically upregulated during cellular stress. Apart from that, it has been shown that hsps are immunodominant. Those unique qualities of hsps (evolutionary conservation, immunodominance and upregulation during stress) have made hsps attractive candidates as targets for immunotherapy and vaccines. As their name implies, HSPs are synthesized by a cell in response to heat shock. To date, three major families of HSPs have been identified based on molecular weight. The families have been called hsp60, hsp70 and hsp90 where the numbers reflect the approximate molecular weight of the stress proteins in kilodaltons. Many members of these families were found subsequently to be induced in response to other stressful stimuli including, but not limited to, nutrient deprivation, metabolic disruption, oxygen radicals, and infection with intracellular pathogens. In certain embodiment the stressful stimuli described hereinabove, may be used to optimize the efficacy of the stressed mononuclear cells used in the methods for treatment described herein.

The term “about” as used herein means in quantitative terms plus or minus 5%, or in another embodiment plus or minus 10%, or in another embodiment plus or minus 15%, or in another embodiment plus or minus 20%.

The term “subject” refers in one embodiment to a mammal including a human in need of therapy for, or susceptible to, a condition or its sequelae. The subject may include dogs, cats, pigs, cows, sheep, goats, horses, rats, and mice and humans. The term “subject” does not exclude an individual that is normal in all respects.

The following examples are presented in order to more fully illustrate the preferred embodiments of the invention. They should in no way be construed, however, as limiting the broad scope of the invention

### Example 1

### Insulin-Dependent Diabetes Mellitus

Basic study design. The effect of a single immunotherapy treatment on postprandial serum glucose levels were evaluated on four independent groups of IDDM patients. Each group differed in the composition of the vaccine administered, as described below under. Before the first treatment, subjects were evaluated for baseline postprandial blood glucose following a standardized meal. Two months following treatment, patients are again evaluated for postprandial glucose levels.

Sixty-eight IDDM patients ranging in age from 12-23 years participated in this study. Diagnoses and long-term management were conducted at Al-Nuzha, Sabah Al-Salem and Amiri Hospital diabetes clinics in Kuwait City. In each case, diagnosis of IDDM was made 3 months or less prior to initiation of immunotherapy. Experimental treatment consent forms were obtained from each individual; or from either the patient or guardian for participants under the age of 18. All patients were informed of the possibility that their treatment might be placebo. Exclusion criteria included pregnancy and serious underlying disease not associated with the diabetic condition. For this study, donors included parents, close relatives or guardians. In the case of the 12 persons in the autologous cell cohort (Group 3, below), the patient's own cells were used. In each case, allogeneic donors were screened for HIV, hepatitis B and C and were required to be in good general health at the time of donation.

Treatment cohorts: Patients were segregated into four groups based on composition of vaccinations to be administered. These included Group 1: 25 patients given live, stressed allogeneic cells; Group 2: 21 patients given heat-killed stressed allogeneic cells; Group 3: 12 patients given live, stressed autologous cells; and Group 4: 10 patients given placebo (Ringers Lactate).

Preparation of donor cells: 150 ml of heparinized blood was collected from each donor, followed by Ficoll-Hypaque density centrifugation to extract peripheral blood mononuclear cells (PBMC). Cells are then resuspended at a density of approximately 1.0×10⁶ cells/ml in RPMI1640 cell culture medium, supplemented with 10% donor serum; and heated in 6 well plates or 500 ml culture flasks to 42 deg C. for forty minutes (stress condition). Next, donor cells (both adherent and non-adherent fractions) are placed in 6 well plates or 500 ml culture flasks for 48 hours at 37 deg C., 5% CO₂. The nonadherent cellular fraction of each culture (mainly lymphocytes); and the adherent (macrophage/monocyte) fraction were harvested then washed 3 times and resuspended in Ringer's Lactate IV in preparation for administration to donors. Cells to be administered to Group 3, were incubated at 50+deg C. to kill the cells. Cell killing was confirmed by Trypan Blue viability assay.

Treatment protocol: Each participating subject was required to fast for 6 hours or more prior to treatment. Upon arrival at the immunotherapy clinic the patient was given a standard meal consisting of 250 grams of rice, 250 grams of mutton and 250 ml of milk. After a 2 hour interval venous blood was drawn for the baseline (pre-therapy) measurement of post-prandial blood sugar. Blood glucose levels were measured in the plasma fraction of each sample using a glucose oxidase-based assay (Glucose Analyzer Ill., Beckman Inc., USA). Results are reported in mmol glucose per liter of whole blood. The cell preparation was vaccinated subcutaneously into the forearms of each participating subject in the treatment group. Vaccinations were administered four per arm, in a pattern such that each injection was approximately 2 cm from the other 3.

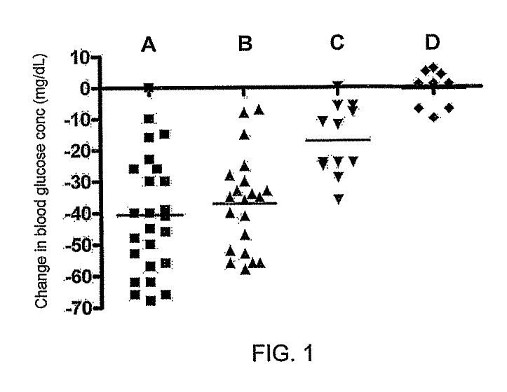

Results: As shown in FIG. 1, outcomes are reported as percentage change in postprandial serum glucose for each subject during the period of time (2 months) between baseline and final measurement of blood sugar. Values were compared with patients treated with a placebo consisting of the injection vehicle (Ringer's Lactate IV). As shown in data set A, patients receiving live, stressed allogeneic cells manifested substantial and uniformly negative changes in post-prandial blood sugar (−39.9±3.77%) 2 months following vaccination; while PPBS levels in placebo-treated patients fluctuated in both directions, with an overall change of 0.117±1.93% (p<0.01). Subjects administered heat-killed leukocytes also experienced uniformly decreased PPBS after therapy, with an average change of −37.1±3.24% versus placebo (p<0.01)(Data set B). Interestingly, patients responded to vaccination with live, stressed autologous cells by uniform decreases in PPBS, showing an average change in this variable of −16.3±3.11% (Data set C), a significantly greater decrease than placebo-vaccinated subjects (p<0.01) (Data set D).

### Example 2

### Rheumatoid Arthritis

Basic design: Two groups of rheumatoid arthritis (RA) patients served as subjects for this study. One group was administered the immunotherapy of the invention and was evaluated for occurrence of the disease activity markers 6 weeks following treatment. Another group of recently-diagnosed RA patients was evaluated for the same biomarkers prior to any treatment.

Erythrocyte sedimentation and serum rheumatoid factor concentrations were evaluated by the nephelometric method with ESR reported in mm/hr and serum RF as IU/mil. The criteria for disease-associated pain were subjective based on the patient's self-evaluation. Presence of pain was designated “1” and absence “0”.

Patients and donors: 200 adult residents of Kuwait diagnosed with rheumatoid arthritis participated in this study. Diagnoses and long-term management were conducted at Mubarak Al-Kabeer and Amiri Hospitals in Kuwait. Exclusion criteria included serious underlying disease not associated with rheumatoid arthritis. All patients receiving immunotherapy discontinued disease-modifying antirheumatic drugs during the 6-week period between treatment and clinical/lab evaluation. Cell donors were primarily spouses and in some cases in some cases included close relatives or friends. In each case donors were screened for HIV, hepatitis B and C and were required to be in good general health at the time of donation.

Treatment cohorts: RA Patients were segregated into two groups based treatment received: Group 1 (treatment group): 100 patients to be administered live, stressed allogeneic cells only; and Group 2 (control): 100 untreated patients.

Out of 100 participants in each group, resources were available for RF and ESR evaluation of only 53 treated and 53 control patients. Individuals to receive these assays were selected randomly. All 100 individuals in both groups were evaluated for pain.

Preparation of donor cells: 150 ml of heparinized blood was collected from each donor, followed by Ficoll-Hypaque density centrifugation to extract the peripheral blood mononuclear cells (PBMC). Cells are then resuspended at a density of approximately 1.0×10⁶ cells/ml in RPMI1640 cell culture medium, supplemented with 10% donor serum; and cultured under stress conditions in 500 ml culture flasks' for 72 hours at approximately 22 deg C., 5% CO2. The nonadherent cellular fraction of each culture (mainly lymphocytes); and the adherent (macrophage/monocyte) fraction were harvested then washed 3 times and resuspended in Ringer's Lactate IV in preparation for administration to donors.

Treatment protocol: The cell preparation was vaccinated subcutaneously into the forearms of each participating subject in the treatment group. Vaccinations were administered four per arm, in a pattern such that each injection was approximately 2 cm from the other 3.

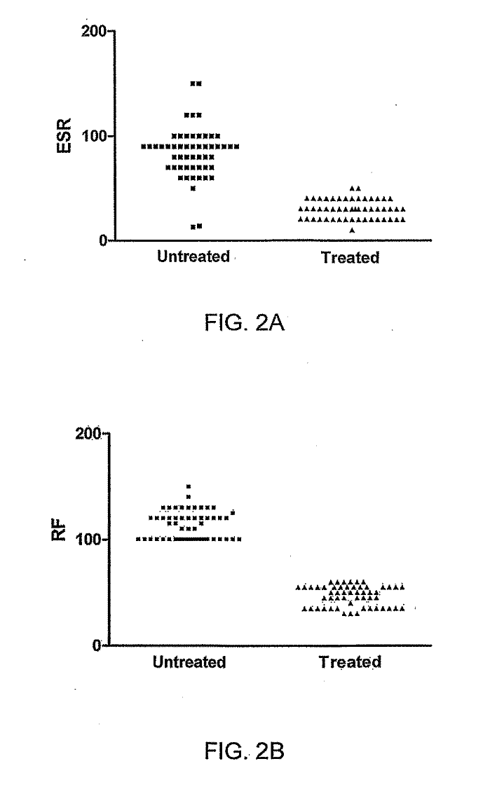

Results: ESR values in both treated and control patients were elevated several-fold in excess of the expected normal range of <20 mm/hr (FIG. 2A). However the mean ESR value for the treated group was lower than the mean of the control group. A value of <60 IU/ml serum RF is expected in a normal population. As shown in FIG. 2B, REF levels in peripheral blood of untreated patients were approximately 2-fold in excess of this level, while subjects receiving immunotherapy all exhibited serum RF levels within the normal range. RF mean values for the treatment group were lower than the untreated group (p<0.05) (FIG. 2B). Comparison of occurrence of self-assessed joint pain in the treated versus untreated cohorts. 97 of the 100 untreated patients reported pain on the day of evaluation, whereas only 4 of the 100 treated patients reported pain on evaluation.

### Example 3

### Uveitis

Basic design. Two groups of uveitis patients served as subjects for this study. One group was administered immunotherapy and was evaluated by slit lamp microscopy for presence of cells in the anterior chamber of the eye. Presence of these cells was designated “1” and absence “0”.

Patients and donors: 225 adult residents of Kuwait diagnosed with uveitis participated in this study. Diagnoses and long-term management were conducted at the Ibn Sina Hospital Eye Clinic in Kuwait. Participants designated to receive immunotherapy were informed of the nature of the treatment and of the study. The untreated control patients were informed that results of their evaluations would be used as data for a study of immunotherapy. Exclusion criteria included serious underlying eye disease not associated with uveitis. All patients receiving immunotherapy discontinued disease-modifying drugs during the 6-week period between treatment and clinical/lab evaluation. Cell donors were primarily spouses and in some cases in some cases included close relatives or friends. In each case donors were screened for HIV, hepatitis B and C and were required to be in good general health at the time of donation.

Treatment cohorts: Uveitis patients were segregated into two groups based treatment received: Group 1 (treatment group): 105 patients to be administered live, stressed allogeneic cells only; Group 2 (control): 120 untreated patients.

Preparation of donor cells: 150 ml of heparinized blood was collected from each donor, followed by Ficoll-Hypaque density centrifugation to extract the peripheral blood mononuclear cells (PBMC). Cells are then resuspended at a density of approximately 1.0×10⁶ cells/ml in RPMI1640 cell culture medium, supplemented with 10% donor serum; and heated in 6 well plates or 500 ml flasks to 42 deg C. for forty minutes (stress condition). Following this step, cells are cultured for 48 hours at 37 deg C., 5% CO₂. The nonadherent cellular fraction of each culture (mainly lymphocytes); and the adherent (macrophage/monocyte) fraction were harvested then washed 3 times and resuspended in Ringer's Lactate IV in preparation for administration to donors.

Treatment protocol: The cell preparation was vaccinated subcutaneously into the forearms of each participating subject in the treatment group. Vaccinations were administered four per arm, in a pattern such that each injection was approximately 2 cm from the other 3.

Results: Comparison of occurrence of leukocyte infiltration in the treated versus untreated cohorts show that 111 of the 120 untreated patients were observed to have significant infiltrate of the iris on the day of evaluation, whereas only 7 of the 105 treated patients exhibited this infiltrate at evaluation.

### Example 4

### Psoriasis and Alopecia

Major clinical features of psoriasis typically include skin lesions resulting from autoimmune attack on keratinocytes. Clinical features of alopecia are principally mild to severe depilation due to autoimmune attack on hair-producing tissue of the follicles.

Basic design Two patients, one afflicted with psoriasis, the other with alopecia served as subjects for this study. No placebo group was included in the present study.

Patients and donors: Psoriasis: A 19 year-old Kuwaiti male, cell donor: brother. Alopecia aereata: 34 year-old Kuwaiti female, cell donor: husband. In each case donors were screened for HIV, hepatitis B and C and were required to be in good general health at the time of donation.

Preparation of donor cells: 150 ml of heparinized blood was collected from each donor, followed by Ficoll-Hypaque density centrifugation to extract the peripheral blood mononuclear cells (PBMC). Cells are then resuspended at a density of approximately 1.0×10⁶ cells/ml in RPMI1640 cell culture medium, supplemented with 10% donor serum; and stressed in 500 ml culture flasks for 72 hours at approximately 22 deg C., 5% CO₂. The nonadherent cellular fraction of each culture (mainly lymphocytes); and the adherent (macrophage/monocyte) fraction were harvested then washed 3 times and resuspended in Ringer's Lactate IV in preparation for administration to donors.

Treatment protocol: The cell preparation was vaccinated subcutaneously into the forearms of each participating subject. Vaccinations were administered four per arm, in a pattern such that each injection was approximately 2 cm from the other 3.

Results: Substantial improvement in prognosis for both patients occurred during the 4-month interval between administration of treatment and following evaluations.

### Example 5

### Case Studies: Uveitis

One patient was a 22 year old Kuwaiti female with bilateral anterior uveitis. She presented with combined follicular and papillary conjunctival reaction (adenoviral keratoconjunctivitis) with ended with active epithelial staining leading to subepithelial infiltrates (scarring), and was put on steroid treatment. Two years later she was then treated in accordance with the invention and subsequently found to have no epithelial staining and only scarring; steroids were stopped. Another two years later, examination showed no corneal pathology, without staining or scarring. Conditions for preparation of the donor cells and their administration to the patient are the same as those described in Example 3 (uveitis), above.

In another case study, a 17-year old patient diagnosed with autoimmune uveitis with immunocompetent cell infiltration was administered immunotherapy as described above three weeks later. Two months after treatment, slit lamp and staining procedures showed no autoimmune disease.

### Example 6

### Other Autoimmune Diseases

According to another embodiment of this invention, treatment is provided to patients suffering from Hashimoto's autoimmune thyroiditis, Ig-nephropathy, pemphigus vulgaris and systemic lupus erythematosus. In all these cases, the disease was either substantially ameliorated or completely cured upon completion of the treatment according to this invention.

The foregoing has been a description of certain non-limiting preferred embodiments of the invention. Those of ordinary skill in the art will appreciate that various changes and modifications to this description may be made without departing from the spirit or scope of the present invention, as defined in the following claims.

What is claimed is:

1. A method for treating an autoimmune disease in a subject comprising the step of: administering to said subject a composition comprising stressed mononuclear cells, wherein said stressed mononuclear cells induce tolerance to self-antigens, thereby treating the autoimmune disease.

2. The method of claim 1, whereby in the step of administering, the stressed mononuclear cells are killed.

3. The method of claim 1 whereby in the step of administering, the stressed mononuclear cells are killed by freezing.

4. The method of claim 1 whereby in the step of administering, the stressed mononuclear cells are autologous or allogeneic.

5. The method of claim 1, whereby in the step of administering, the mononuclear cells are obtained from a mixed mononuclear cell population.

6. The method of claim 5 wherein the mixed mononuclear cell population is obtained from a subject, a pool of subjects, a cell line or a combination thereof.

7. The method of claim 1 wherein the mononuclear cells are maintained in culture ex-vivo prior to being stressed, after being stressed, of the combination thereof.

8. The method of claim 7, wherein said culture is maintained between about 37° C.

9. The method of claim 7, wherein said culture is maintained between about 20 to 80 hours.

10. The method of claim 1, wherein the stressed mononuclear cells are stressed by exposure of said mononuclear cells to a temperature between about 40 to about 50° C., for between about 20 minutes to about 36 hours.

11. The method of claim 10 wherein the temperature is between about 40 to about 45° C., for about 30 minutes to about 1 hour.

12. The method of claim, 11 wherein the temperature is about 42° C. for about 40 minutes,

13. The method of claim 1, wherein the stressed mononuclear cells are stressed by exposure of said mononuclear cells to a temperature between about 20 to about 25° C., for between about 24 to about 72 hours.

14. The method of claim 7, wherein said culture is maintained at a temperature of about 37° C. for about 24 hours, and the mononuclear cells are stressed at a temperature of about 4° C. for about 24 hours.

15. The method of claim 1, wherein said step of administering is by a subcutaneous, an intradermal, an intraperitoneal route, or a combination thereof.

16. The method of claim 1 wherein the cells are administered to the subject at four to eight weekly intervals.

17. The method of claim 16, comprising 3-4 administrations.

18. The method of claim 1 wherein the autoimmune diseases is conventional organ specific autoimmunity disease, neurological disease, rheumatic diseases, connective tissue disease, autoimmune cytopenia, or related autoimmune disease and their combination.

19. The method of claim 18 wherein the conventional organ specific autoimmunity is thyroiditis, gastritis, adrenalitis (Addison's), ovaritis, primary biliary cirrhosis, myasthenia gravis, gonadal failure, hypoparathyroidism, alopecia, malabsorption syndrome, pernicious anemia, hepatitis, anti-receptor antibody diseases, hypopituitarism, diabetes insipidus, sicca syndrome or a combination thereof.

20. The method of claim 18, wherein the neurological disease is multiple sclerosis or chronic inflammatory demyelinating polyradiculoneuropathy.

21. The method of claim 18, wherein the rheumatic diseases or connective tissue diseases is rheumatoid arthritis, systemic lupus erythematous (SLE) or lupus, scleroderma, Reiter's syndrome, polymyositis, inflammatory bowel disease, dermatomyositis, ulcerative colitis, Crohn's disease, vasculitis, psoriatic arthritis, exfoliative psoriatic dermatitis, psoriasis, pemphigus vulgaris, Sjorgren's syndrome, or a combination thereof.

22. The method of claim 1 wherein the autoimmune diseases is autoimmune uveitis, glomerulonephritis, post myocardial infarction cardiotomy syndrome, pulmonary hemosiderosis, amyloidosis, sarcoidosis, aphthous stomatitis or a combination thereof.

23. A method for treating Diabetes in a subject, comprising administering to said subject a composition comprising thermally stressed mononuclear cells, wherein said thermally stressed mononuclear cells induce tolerance to self antigens.

24. The method of claim 23, wherein said thermal stress comprises exposing the mononuclear cells to temperature of between about 40 to about 45° C., for a period of about 30 minutes.

25. A method for treating rheumatoid arthritis (RA), psoriasis or their combination in a subject, comprising administering to said subject a composition comprising thermally stressed mononuclear cells, wherein said thermally stressed mononuclear cells induce tolerance to self antigens.

26. The method of claim 25, wherein said thermal stress comprises exposing the mononuclear cells to temperature of between about 20 to about 25° C., for a period of about 72 hours.

27. A method for treating uveitis in a subject, comprising administering to said subject a composition comprising thermally stressed mononuclear cells, wherein said thermally stressed mononuclear cells induce tolerance to self antigens.

28. The method of claim 27, wherein said thermal stress comprises exposing the mononuclear cells to temperature of between about 40 to about 45° C., for a period of about 40 minutes, followed by 48 hours at 37° C.

29. A vaccine for the treatment of an autoimmune disease comprising thermally stressed mononuclear cells, wherein said thermally stressed mononuclear cells induce tolerance to self-antigens.
